(19) United States
(12) Patent Application Publication
(10) Pub. No.: US 2024/0325088 A1
(43) Pub. Date: Oct. 3, 2024

(54) Augmented Reality-Driven Guidance for Interventional Procedures

(71) Applicant: The Medical College of Wisconsin, Inc., Milwaukee, WI (US)

(72) Inventors: Eric Scott Paulson, Jackson, WI (US); Nikolai J. Mickevicius, Milwaukee, WI (US)

(21) Appl. No.: 18/578,948

(22) PCT Filed: Jul. 12, 2022
(86) PCT No.: PCT/US2022/036869

§ 371 Date: Jan. 12, 2024

Related U.S. Application Data
(60) Provisional application No. 63/220,921, filed on Jul. 12, 2021.

(51) Int. Cl. A61B 34/20; A61B 90/00; A61N 5/10; G01R 33/561

(52) U.S. Cl. CPC A61B 34/20; A61B 90/37; A61N 5/1007; A61N 5/1049; G01R 33/5615; A61B 2034/2051; A61B 2090/365; A61B 2090/372; A61N 2005/1055

(57) ABSTRACT

Described here are systems and methods for guiding interventional procedures, in which the guidance is driven by a virtual reality, augmented reality, augmented virtuality, and/or other mixed reality systems. The virtual/mixed reality guidance can be based on magnetic resonance images that are acquired in real-time and presented to a user in a virtual/mixed reality environment.

### BACKGROUND

In physician-driven interventions, physicians are required to view external monitors during procedures. This is disadvantageous, as the physician has to take his/her eyes off the patient during the procedure in order to view the external monitor.

### SUMMARY OF THE DISCLOSURE

The present disclosure addresses the aforementioned drawbacks by providing a method for image-guided alignment of an interventional device. Medical image data are accessed with a computer system, where the medical image data are acquired from a subject in real-time with a medical imaging system. The medical image data depict an anatomical target. An augmented reality environment is generated with the computer system based in part on the medical image data. The augmented reality environment includes at least one visual guide indicating a separation distance between a reference location and the anatomical target. Generating the augmented reality environment includes overlaying the at least one visual guide with a view of the subject in a real-world environment. The augmented reality environment is displayed to a user using a display while medical image data continue to be acquired in real-time from the subject with the medical imaging system. Via the at least one visual guide in the augmented reality environment, an indication is generated when the reference location is aligned with the anatomical target.

It is another aspect of the present disclosure to provide a method for controlling the delivery of radiation treatment. Medical images of a subject are accessed with a computer system, wherein the medical images are acquired in real-time with a medical imaging system, wherein the medical images depict an anatomical target. Treatment contour data are also accessed with the computer system. A virtual reality environment is generated with the computer system using the medical images and the treatment contour data. The virtual reality environment depicts a scene in which the treatment contour data are overlaid with the medical images. The virtual reality environment is displayed to the subject using a display while medical images continue to be acquired in real-time from the subject with the medical imaging system. A radiation treatment system is triggered to turn on a radiation beam when the anatomical target is aligned within a contour of the treatment contour data within the virtual environment.

It is another aspect of the present disclosure to provide a method for image-guided alignment of an interventional device. Medical images of a subject are accessed with a computer system, wherein the medical images are acquired in real-time with a medical imaging system, wherein the medical images depict an anatomical target. An augmented reality environment is generated with the computer system using the medical images, and may also depict an interventional device and/or a surrogate of the interventional device. The augmented reality environment depicts a scene in which the medical images are overlaid with a view of the subject in a real-world environment. The augmented reality environment is displayed to a user using a display while medical images continue to be acquired in real-time from the subject with the medical imaging system. Based on the augmented reality environment, an interventional device is aligned with the anatomical target.

It is another aspect of the present disclosure to provide a method for aligning a subject with a radiation beam of a radiation treatment system. Patient model data are accessed with a computer system. An augmented reality environment is generated with the computer system using the patient model data, wherein the augmented reality environment depicts a scene in which the patient model data are overlaid with a real-world environment. The augmented reality environment is displayed to a user using a display. An indication is generated in the augmented reality environment when the patient model is aligned with a radiation beam of a radiation treatment system within the scene.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a flowchart setting forth the steps of an example method for generating a virtual and/or augmented reality environment in accordance with some embodiments described in the present disclosure.

FIG. 2 is an example of a head-mounted display (“HMD”) that can be implemented in accordance with some embodiments described in the present disclosure.

FIG. 3 illustrates an example augmented reality environment, or scene, in which visual guides that indicate the separation distances between a reference location and a target location in three orthogonal spatial dimensions (e.g., the anterior-posterior dimension, the right-left dimension, the superior-inferior dimension) are generated, displayed, and updated as the positions of the reference and target locations change.

FIG. 4 illustrates an example pulse sequence that can be implemented to acquire both tracking and imaging data using a magnetic resonance imaging (“MRI”) system.

FIG. 5 shows an example of a system including one or more head-mounted displays and various computing devices that can be used to present medical imaging data and related images or data to a user in an interactive virtual reality and/or augmented reality environment.

FIG. 6 shows example components that can implement the system shown in FIG. 5.

FIG. 7 is a block diagram of an example MRI system, which as shown may in some instances be implemented as a combined MRI linear accelerator (“MR-Linac”) system.

### DETAILED DESCRIPTION

Described here are systems and methods for guiding interventional procedures using magnetic resonance imaging (“MRI”), in which the guidance is driven by a virtual reality, augmented reality, augmented virtuality, and/or other mixed reality systems.

Virtual reality (“VR”) provides a computer-simulated virtual environment where a user can interact with the virtual environment or virtual objects in that virtual environment. A user is able to interact with the virtual environment through the use of one or more user input devices.

Augmented reality (“AR”) provides a live view of a real-world environment that is augmented by computer-generated virtual objects. This augmentation can be provided in real-time. Like VR, a user is able to interact with the virtual objects in this augmented reality environment through the use of one or more user input devices. In generating the augmented reality environment, a computer system determines the position and orientation of one or more virtual objects to be displayed to a user in order to augment the real-world environment. These augmentation methods can implement a marker-based recognition or a markerless-based recognition.

In marker-based recognition, one or more markers (e.g., fiducial markers, radio frequency (“RF”) tracking coils) positioned in the real-world environment are identified, detected, or otherwise recognized in an image. A virtual object can then be generated and placed at, or in a location relative to, a detected marker. Additionally or alternatively, the virtual object can include one or more guides presented to the user and indicating positional information about the one or more markers. For example, the virtual object can include one or more guides that are presented to the user and each indicate a relative distance between a reference point, or object, and one or more markers. In markerless-based recognition, the virtual objects are generated and placed at locations in the augmented reality environment based on patterns, colors, or other features detected in an image.

Augmented virtuality (“AV”) is another form of mixed reality, and is similar to AR. In AV, a virtual environment is provided to a user and augmented with physical objects, which may include people. The physical objects can be dynamically integrated into the virtual environment, and can interact with this virtual environment in real-time.

For the sake of simplicity, the term “virtual reality” is used herein to refer to virtual reality, augmented reality, augmented virtuality, and/or other mixed reality systems.

In general, the systems and methods described in the present disclosure use magnetic resonance image data (e.g., magnetic resonance images) to generate virtual objects and/or a virtual environment for displaying in a virtual reality, augmented reality, augmented virtuality, and/or other mixed reality environment.

In some aspects, the systems and methods described in the present disclosure provide for physician-driven applications, in which a physician or other clinician is the user of the virtual reality system. In some other aspects, the systems and methods described in the present disclosure provide for patient-driven applications, in which a patient is the user of the virtual reality system. In still other aspects, the systems and methods described in the present disclosure provide for treatment team-driven applications, in which one or more members of a treatment team (e.g., surgical team, radiation treatment team) is a user of the virtual reality system.

In physician-driven applications, real-time information can be displayed to the user (i.e., a physician) in order to help guide the user during an image-guided procedure. Advantageously, in these applications the treatment time, tissue damage to the patient, or both, can be reduced. As one example, real-time information that can be displayed to the user can include static serial or real-time magnetic resonance images acquired from the patient and displayed to the user in real-time. Additionally or alternatively, the real-time information that can be displayed to the user can include physiological data (e.g., patient vital signs) that are acquired from the patient and displayed to the user in real-time. In still other instances, the real-time information that can be displayed to the user can include one or more visual guides that indicate positional information about a target location. For instance, visual guides can be generated and displayed to the user in order to indicate a relative distance between a target location (e.g., a tumor, a treatment zone) and a reference location (e.g., a medical device, a prescribed radiation beam path or focal region).

Non-limiting examples of physician-driven applications can include MR-guided interstitial brachytherapy, MR-guided biopsies (e.g., breast, prostate), MR-guided cardiac ablation, MR-guided catheterization, and MR-guided paravertebral sympathetic injections. In each of these applications, the virtual reality system can display magnetic resonance images obtained from the patient in real-time to the user. These images can be displayed in a virtual environment, or in an augmented reality environment. For instance, the images can be registered with the real-world environment (e.g., using a marker-based recognition or a markerless-based recognition) such that the images, or portions thereof, are visually overlaid on the patient when the user is looking at the patient. In this way, the user can simultaneously visualize the surgical tool (e.g., brachytherapy seed needle, biopsy needle, injector needle) relative to the patient's internal and external anatomy.

Additionally or alternatively, one or more visual guides can be displayed in the virtual environment, or in an augmented reality environment. For instance, visual guides can indicate relative distances between two points or objects (e.g., a target location and a reference location). As a non-limiting example, the visual guides can include a first visual guide indicating a relative distance along a first spatial dimension and a second visual guide indicating a relative distance along a second spatial dimension that is preferably orthogonal to the first spatial dimension. In some implementations, a third visual guide can also be displayed, where the third visual guide indicates a relative distance along a third spatial dimension, which may preferably be orthogonal to both the first and second spatial dimensions. As an example, the visual guides can include guides that indicate a relative measure of anterior-posterior separation, right-left separation, and/or superior-inferior separation between a target location and a reference location. The visual guides can be displayed in the periphery of the virtual environment presented to the user, providing an unobtrusive indication of important positional information during an interventional procedure.

In these applications, the user does not need to take their eyes off of the patient during the procedure in order to view images on an external monitor. Advantageously, using an augmented reality environment can enable more intuitive guidance compared to existing methods. For instance, in these applications physicians do not need to mentally rotate coordinate systems when performing procedures, as is often necessary when using current methods that utilize external monitors. Instead, by displaying image overlays and/or visual guides that indicate relative positional information, the user is able to visualize pertinent information (e.g., images of anatomy, proximity between two spatial points) within the context of the real-world position and orientation of the patient.

As another advantage, when using a HMD that includes a camera, photographs can be taken during a procedure for documentation (e.g., for medical documentation of the procedure).

Accelerometers in HMDs can also be used to drive real-time information changes and to enable user interaction with the virtual reality or augmented reality environment. For example, left/right head rotation of the user could scroll through pages of real-time information (e.g., medical records, pre-surgical images, physiological data).

Additionally or alternatively, head rotation of the user could control real-time prescription of cine MR imaging planes to optimize visualization. In this way, the virtual reality system can interface with and provide feedback to the MRI system in order to control the scan prescription (e.g., pulse sequence parameter selection).

Additionally or alternatively, head rotation of the user could control the transparency of the magnetic resonance images being overlaid on the user's view of the real-world environment. For instance, the user could control the degree of transparency of these images in order to emphasize or deemphasize this information during the procedure, as desired.

In patient-driven applications, real-time information can be displayed to the patient, enabling the patient to actively engage with the treatment team to achieve the best possible treatment outcome. For instance, in these applications the patient can be provided with real-time information that enables the patient to adapt their position or internal anatomy in order to improve the efficacy and safety of the treatment delivery.

As one example, real-time information that can be displayed to the user can include magnetic resonance images acquired from the patient and displayed to the user in real-time. These images can include images depicting internal anatomy. Additionally or alternatively these images can include targets obtained from MR-guided radiotherapy.

In these applications, the virtual reality system can provide a virtual environment to the patient. In this virtual environment, the user can be shown magnetic resonance images in real-time as they are being acquired using an MRI system. One or more contours can be displayed on conjunction with the magnetic resonance images, such as by overlaying the one or more contours onto the magnetic resonance images being displayed to the patient. The one or more contours may include contours associated with gross tumor volume (“GTV”), clinical target volume (“CTV”), planning target volume (“PTV”), an organ-at-risk (“OAR”) or multiple OARs, a planning organ-at-risk volume (“PRV”), or other such contours that may be common in radiation treatment planning. Additionally or alternatively, the user can be presented with simplified visual guides that indicate a relative position between two locations of interest, such as a target location (e.g., location of internal anatomy of interest, a treatment zone) and a reference location (e.g., location of a prescribed radiation treatment beam path, location of a medical device or planned medical device trajectory).

As a non-limiting example, the patient can be presented with a virtual environment, or augmented reality environment, that includes continually updated magnetic resonance images as they are being acquired in real-time, onto which a treatment contour, such as a PTV contour, is overlaid.

As the patient is instructed to hold their breath for delivery of radiation treatment, the virtual environment allows the patient to see in real-time whether the appropriate anatomical target is within the contour displayed in the virtual environment. Additionally or alternatively, the patient can be presented with more simplified visual guides that indicate whether the anatomical target is within the prescribed treatment contour(s). This enables the patient to adjust their breath-hold (e.g., by increasing or decreasing inspiration) in order to align the anatomical target within the contour.

In some implementations, one or more contours associated with one or more OARs can also be presented to the patient in the virtual environment. In this way, the patient can adjust their breath-hold in order to align the anatomical target within the PTV contour while also keeping the one or more OARs outside of the PTV contour to which the radiation beam will deliver radiation. Similarly, the patient can additionally or alternatively be presented with one or more visual guides that indicate whether the one or more OARs are outside of the radiation beam path and/or prescribed radiation beam path.

Additionally or alternatively, real-time information that can be displayed to the user can include respiratory signals obtained from internal MRI-based navigators, respiratory bellows, or other suitable means. In these instances, the respiratory signal data can be displayed to the user in real-time in order to enable the user to adjust their respiratory pattern in such a way so as to reduce the time of respiratory-triggered/gated MRI acquisitions. Such data could be presented to a user in a virtual environment or an augmented reality environment. In other implementations, the respiratory signal data can be processed to generate simplified visual guides that are presented to the patient in the virtual or augmented reality environment. These visual guides may indicate, as an example, the relative position of a target location (e.g., an anatomical target) relative to a reference location (e.g., a radiation beam path, a radiation treatment contour). As the patient is breathing, the visual guides can be updated in real-time to indicate how respiration is affecting the positioning of the target location relative to the reference location. In this way, the user can be provided with feedback on whether, and how, to control breathing to better align the target and reference locations.

Additionally or alternatively, real-time information that can be displayed to the user can include other images acquired with an MR-guided high frequency ultrasound (“HIFU”) system.

Additionally or alternatively, real-time information that can be displayed to the user can include real-time images of the patient's surface obtained from a system such as a surface-guided radiotherapy system, in which stereo vision images are used to track the patient's surface in three dimensions. In these applications, treatment times of deep inspiration breath-hold radiotherapy can be reduced.

Additionally or alternatively, real-time information that can be displayed to the user can include real-time video feed of family members or treatment team members to reduce patient anxiety, whether during imaging or treatment delivery.

Additionally or alternatively, real-time information that can be displayed to the user can include data that provide a visual feedback to the patient regarding the imaging scan being conducted or to provide an awareness of the patient's motion. For instance, the real-time information may include a countdown timer indicating the amount of scan time remaining for a given MRI pulse sequence. As another example, the real-time information may include a countdown timer indicating the amount of time remaining for a breath-hold for a given MRI pulse sequence, radiation beam delivery, or both. As still another example, the real-time information may include a message or other indication to the patient informing them not to move. In these applications, the frequency of repeated scans may be reduced because the patient is informed of their movement during the scan, rather than at the end of the scan.

In some implementations, data collected by the virtual reality system used by the patient (e.g., the HMD, sensors embedded in or coupled to the HMD) can also be collected as used for post-processing of images or other data acquired from the patient. For instance, quantitative information about the motion of the patient can be measured from motion sensors in the HMD worn by the patient, and these data can be used during or after image reconstruction to compensate for the motion occurring while the image data were acquired from the patient.

In treatment team-driven applications, real-time information can be displayed to one or more members of the treatment team, such as to ensure that treatment is administered safely, correctly, and efficiently to a patient.

For instance, reviewable sentinel events occur when radiation is delivered to the wrong region or with greater than 25% of the planned dose. These situations can occur if the patient is shifted incorrectly based on daily imaging. To prevent these errors, radiation therapists can wear HMDs in the treatment room. A virtual model of the patient can be projected in space at the treatment isocenter. The radiation therapists would align the actual patient to the virtual model, confirming that any shifts are made in the correct directions. Additionally or alternatively, one or more visual guides can be displayed to a radiation therapist to indicate the relative positioning between a target location in the patient (e.g., a prescribed PTV) and a reference location (e.g., the treatment isocenter). These simplified visual guides can indicate when the target location is properly aligned with the reference location without the need to generating and displaying a more complex patient model. The visual guides may additionally or alternatively provide feedback on the alignment of the treatment isocenter with other target locations. For example, a first set of visual guides may indicate relative positions between a PTV and the treatment isocenter, whereas a second set of visual guides may indicate relative positions between one or more OARs and the treatment isocenter. In this way, the radiation therapist can be provided with feedback not only about whether the PTV is properly aligned with the treatment isocenter, but also whether one or more OARs will be in the radiation beam path.

As another example, the systems and methods described in the present disclosure can enable laser-free setup for radiotherapy patients. Currently, radiation therapists align radiotherapy patients using external lasers and skin tattoos. Similar to the above, with HMDs, radiation therapists could align patients to a virtual model of the patient placed at the treatment isocenter.

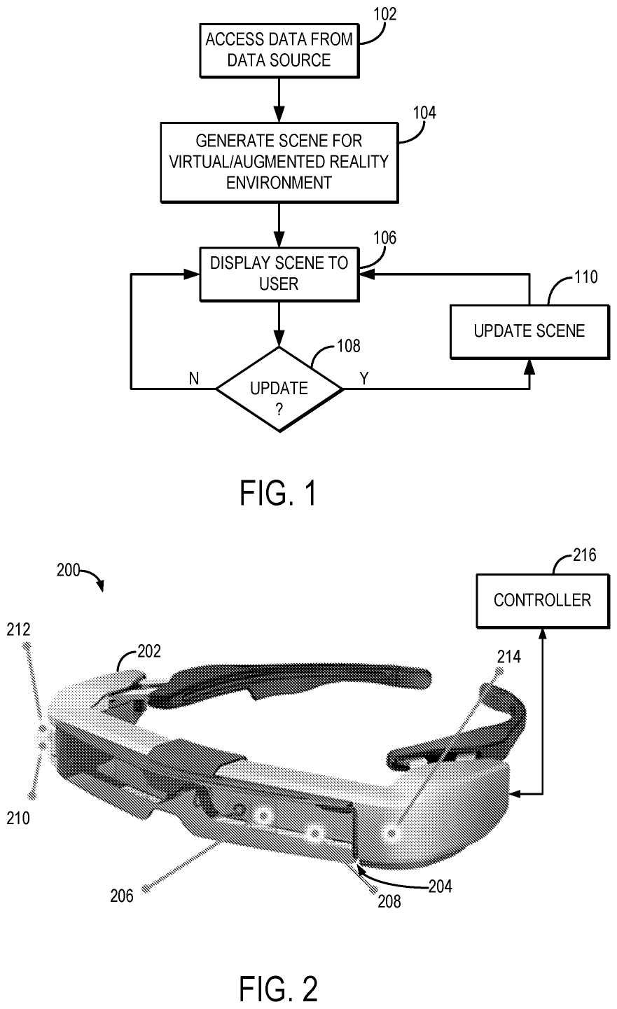

Referring to FIG. 1, a flowchart is illustrated as setting forth the steps of an example method for generating a virtual/augmented reality environment and/or scene for display to a user based on magnetic resonance images and associated data in order to facilitate guidance of a treatment, such as radiation treatment, a surgical procedure, or other interventional procedure.

The method includes assessing data with a computer system, as indicated at step 102. The data can be accessed by retrieving such data from a memory or other suitable data storage device or medium. In other instances, the data can be accessed by acquiring or otherwise obtaining such data and communicating the data to the computer system in real-time from the associated measurement device (e.g., imaging system, physiological measurement device, patient monitor). As described above, the data may be medical imaging data (e.g., magnetic resonance images), physiological data (e.g., respiratory signals, cardiac signals, other patient monitor signals), or other associated data (e.g., patient models, surgical tool models, radiation treatment plan data, treatment system data or parameters). As described above, the computer system can include a computing device, a server, a head-mounted display, or other suitable computer system.

Based on the data, a scene for a virtual and/or augmented reality environment is generated, as indicated at step 104. Generating the scene can include generating any virtual objects for display in the scene, which may include patient models, surgical tool models, treatment plan contour models, physiological data, patient monitor data, or display elements depicting or otherwise associated with such models or data. The one or more virtual models can be arranged in the scene based on information associated with their relative positions and orientations. For instance, the virtual objects can be arranged in the scene based on their location(s) determined using a marker-based or markerless-based recognition.

The scene is then displayed to a user via a head-mounted display or other suitable display device, as indicated at step 106. For instance, the scene can be displayed to the user using a head-mounted display device, in which the generated scene augments the real-world environment, thereby presenting an augmented reality environment to the user. In other instances, the scene can be displayed to the user without overlaying the virtual objects on the real-world environment, thereby providing a virtual reality environment to the user.

A determination is then made at decision block 108 whether any updates should be processed. For instance, if the position or orientation of the scene, the user's view of the scene, and/or virtual or real objects within the scene have changed then appropriate updates to the scene can be made at step 110 and the updated scene displayed to the user at step 106. In other instances, the user may interact with the virtual/augmented reality environment to initiate a change or update to the scene. For example, the user may interact with a real-world or virtual object, which may initiate a change in the scene. As another example, the user may change one or more setting (e.g., display settings) to update the scene (e.g., changing the transparency of a virtual object's display element).

In some applications, user interaction with the scene can be used as feedback for controlling other systems. For example, user interaction with the scene can provide feedback to a radiation treatment system, such that radiation is only delivered when the user (i.e., the patient) satisfies a criterion within the virtual/augmented reality environment (e.g., aligning a portion of their anatomy with a prescribed treatment contour).

Referring to FIG. 2, an example of a head-mounted display (“HMD”) 200 is shown. The HMD 200 can generally take the form of eyeglasses, goggles, or other such eyewear. As one non-limiting example, the HMD 200 can be smart glasses (e.g., the MOVERIO BT-35E; Seiko Epson Corporation, Japan).

The HMD 200 generally includes a frame 202 that defines an opening 204 in which one or more displays 206 are mounted. In the configuration shown in FIG. 2, a see-through window 208, which in some instances may include a single lens or two separate lenses, is mounted in the opened 204 and the one or more displays 206 are embedded in the window 208. In some configurations, a single display 206 is embedded in the window, providing for monocular viewing. In some other configurations, two displays 206 may be embedded in the window 208 to provide for binocular viewing.

The window 208 and each display 206 are at least partially transparent, such that the user can still view the real-world environment when wearing the HMD 200. The window 208 may also be a flip-up style window that can be flipped up and out of the user's line-of-sight when not being used.

In some other configurations, the opening 204 is configured to receive a display 206 that is not transparent, such that the user is not able to view the real-world environment when wearing the HMD 200. In these instances, the HMD 200 can provide a more fully immersive virtual environment to the user. As one example, the opening 204 may be configured to receive a smart phone or other such device.

A camera 210 can be mounted or otherwise coupled to the frame 202 and can be used to obtain images of the real-world environment. These images can be accessed by a computer system, which may in some instances be embedded within the HMD 200 or may be remote to the HMD 200, and used when generating a virtual environment, augmented reality environment, augmented virtuality environment, and/or mixed reality environment, as described below in more detail.

An ambient light sensor 212 can also be mounted or otherwise coupled to the frame 202 and can be used to measure ambient light. Measurements of the ambient light in the real-world environment can be used to adjust the scene presented to the user via the display(s) 206. For instance, the brightness, contrast, color temperature, and/or other image or display settings based on the ambient light in the real-world environment.

One or more motion sensors 214 can also be embedded within or otherwise coupled to the frame 202. The motion sensor(s) 214 measure motion of the HMD 200 and these motion data can be accessed by a computer system, which may in some instances be embedded within the HMD 200 or may be remote to the HMD 200, and used when generating a virtual environment, augmented reality environment, augmented virtuality environment, and/or mixed reality environment. The motion sensor(s) 214 can include one or more accelerometers, gyroscopes, electronic compasses, magnetometers, and combinations thereof.

A controller 216 can interface with the frame 202 to provide video input to the display(s) 206, receive output from the camera 210, ambient light sensor 212, and/or motion sensor(s) 214, and to provide power to the components in or otherwise coupled to the frame 202.

As described above, in some embodiments, a virtual environment and/or augmented reality environment is generated and displayed to a user, in which one or more visual guides are generated and displayed to indicate a relative distance between a reference location and one or more target locations. The reference location can be a location on a medical device (e.g., a catheter tip, a brachytherapy needle tip), the target isocenter of a radiation treatment system, or the like. The target location(s) can include an anatomical target (e.g., a tumor), a planned treatment volume, one or more organs-at-risk, a fiducial marker, and so on.

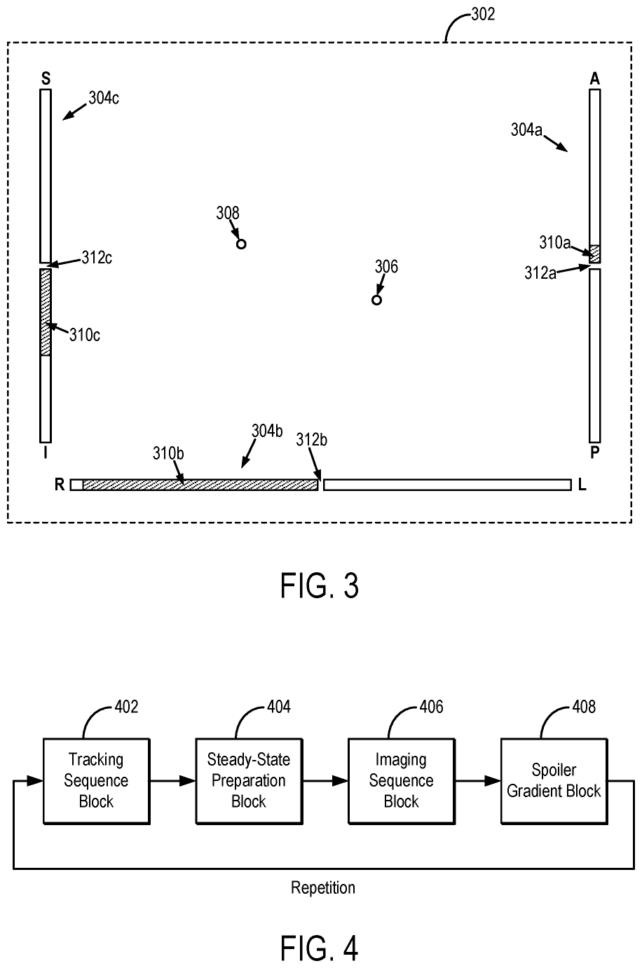

An example of a virtual environment in which visual guides are generated and displayed is shown in FIG. 3. In this example, a scene 302 is displayed to a user, such as via a heads-up display or other such display device. The scene 302 may include an augmented reality scene in which one or more virtual objects (e.g., the visual guides) are presented as an overlay on the real-world environment. The scene 302 may alternatively include a virtual reality scene. One or more visual guides 304 are generated and displayed as an overlay on the scene 302.

For example, the visual guides 304 can include a first visual guide 304a indicating a relative distance between a reference location 306 and a target location 308 along a first spatial dimension, a second visual guide 304b indicating a relative distance between the reference location 306 and the target location 308 along a second spatial dimension, and a third visual guide 304c indicating a relative distance between the reference location 306 and the target location 308 along a third spatial dimension. The first, second, and third spatial dimensions may all be orthogonal. As illustrated, the first spatial dimension corresponds to the anterior-superior dimension, the second spatial dimension correspond to the right-left dimension, and the third spatial dimension correspond to the superior-inferior dimension.

In the illustrated embodiment, each visual guide 304 indicates the relative distance of the reference location 306 from the target location 308 by generating and displaying a visual element that indicates a magnitude of separation distance between the reference location 306 and the target location 308 along the respective spatial dimension. By way of example, when the reference location 306 is anterior to the target location 308, the first visual guide 304a will indicate this separation distance by generating a linear element 310a that shows the target location 308 is anterior to the reference location 308. The length of the linear element 310a provides a visual indication of how far anterior the target location 308 is from the reference location 306. The center 312a of the first visual guide 304a indicates the position where the reference location 306 and target location 308 are aligned along the first spatial dimension. Similarly, linear elements 310b and 312c indicate the relative distance along the second and third spatial dimensions, respectively, with the centers 312b and 312c of the second and third visual guides 304b, 304c, respectively, indicating the positions where the reference location n306 and target location 308 are aligned along the second and third spatial dimensions, respectively.

To improve the visibility of the visual guides 304, the linear elements 310 can be colored. For instance, the linear elements 310 may be colored based on the magnitude of the separation distance along each spatial dimension. A small separation distance may be color-coded as green, an intermediate separation distance may be color-coded as yellow, and a large separation distance may be color-coded as red. The small, intermediate, and large separation distance thresholds can be determined or set based on the scale and/or tolerances of the task at hand.

As one example, the small separation distance may be set as separation distances less than 0.5 cm, the intermediate separation distance may be set as separation distances between 0.5 and 1 cm, and the larger separation distance may be set as separation distances greater than 1 cm. As another example, the small, intermediate, and large separation distance thresholds can be set as a percentage of a maximal separation distance associated with the task at hand. The maximal separation distance may be established relative to the field-of-view of the MRI scanner, the patient anatomy, or the like. Additionally or alternatively, the maximal separation distance may be set as a user selected quantity. The percentages may be set by the user, or established relative to the acceptable tolerances for the clinical task. For example, the small separation distance threshold may be set as up to between 10⁻³³% of the maximal separation distance; the large separation distance threshold may be set as over between 67-100% of the maximal separation distance; and the intermediate separation distance threshold may be set based on those separation distances between the small and large ranges.

Having the linear elements 310 be colored can improve their visibility, especially when the visual guides 304 are generated and displayed in the periphery of the scene 302. In these instances, the colored linear elements 310 may be discernable with the user's peripheral vision, allowing for the user to track the separation distance between the reference location 306 and the target location 308 without having to divert their eyes from the task at hand.

In some embodiments, one or more additional visual elements can be generated and displayed to indicate a tolerance or threshold separation distance along one or more of the spatial dimensions. For instance, a tolerance indicator can be generated to indicate an acceptable tolerance of separation between the reference location 306 and target location 308 along one or more of the spatial dimensions.

As an example, tolerance bars can be overlaid on the visual guides 304 to demarcate a central region on the guide indicating where the reference location 306 and target location 308 are sufficiently aligned. These tolerance bars may be adjustable by the user depending on the task at hand and the acceptable clinical tolerances. In some embodiments, the tolerances may be established by a radiation treatment plan. For instance, the tolerances may be associated with acceptable treatment margins, locations of OARs, and so on.

As another example, the center 312 of each visual guide 304 may be sized to match the acceptable tolerances for separation distance. For instance, the center 312 may be sized such that the linear elements 310 are not generated and displayed until the distance between the reference location 306 and target location 308 is larger than the distance indicated by the size of the center 312.

In an example implementation, real-time catheter position data and target images from a tracking/imaging sequence can be sent to a computer workstation using the OpenIGTLink protocol. The computer workstation can be placed on the local MRI network to minimize communication latency and may be physically positioned inside the MRI scanner room, enabling it to be the interfaced to the AR headset. Target contours, drawn on a reference image prior to catheter insertion can be deformably propagated to the latest 3D target image received over OpenIGTLink communication using an optical flow algorithm. In addition to targets, the HUD software could also incorporate healthy organ contours (e.g., bladder, sigmoid, rectum) that should be avoided during insertion, Differences between catheter position and updated target centroid positions along the three cardinal axes can be calculated and displayed in a HUD. The HUD will be projected to the AR headset worn by the user (e.g., radiation oncologist) in the MRI bore during interventional procedures.

Additionally or alternatively, the HMD can display textual information in the scene 302, such as position deltas between the reference location 306 and the target location 308. For example, the reference location 306 can be tracked relative to the target location 308. A target contour can be deformed and position deltas determined. The position deltas can then be displayed to the user.

In some implementations where an interventional procedure is being performed with a medical device that is being tracked in real-time (e.g., a catheter, a brachytherapy needle), a location of the medical device can be tracked using one or more tracking RF coils that are coupled to the medical device.

As a non-limiting example, active catheter tracking can be implemented using micro RF coils integrated within the tips of tungsten obturators inserted into the catheter lumen during navigation. In an example implementation, four-loop planar rectangular micro RF receive coils (approximately 1.5 mm×8.0 mm) can be used for tracking. The micro RF coils can be fabricated using dual-sided flexible printed circuit sheets.

In an example embodiment, the micro RF coils can be coupled to obturators, such as commercially available tungsten obturators. Slots and grooves can be machined into the distal end of the obturators to accommodate the coils and micro coaxial cables, respectively. Two micro RF coils can be attached to the distal slots of each obturator. The micro coaxial cables run through the groove in the obturator and can interface with an isolation box at the proximal end of the obturator. The isolation box can contain patient electrical isolation and coil tuning-matching and decoupling circuitry. The distal end of the isolation box can contain a coaxial cable that interfaces to two MRI receivers using a standard coil plug. Dedicated RF coil files can be programmed to enable interfacing of the micro RF coils with the MRI scanner software, facilitating simultaneous selection of micro RF coils along with standard imaging coils during acquisition.

Additionally or alternatively, wirelessly connected resonance circuits (“wRC”) can be used in lieu of micro RF coils. Advantageously, wRCs do not utilize dedicated connections to MRI receivers or interfacing with MRI scanner software and, thus, may be simpler to implement.

When using tracking RF coils, the data acquisition of the MRI system can be adapted to acquire data that facilitate the real-time tracking of objects during a surgical or other interventional procedure. For example, the MRI system can be operated using a pulse sequence that is designed to simultaneously track a reference location (e.g., a catheter tip, brachytherapy needle) and one or more target locations (e.g., anatomical target(s), treatment zone(s)).

As a non-limiting example, due to tissue deformation during catheter insertion, active tracking of a catheter tip alone may not be sufficient to guide accurate positioning of catheters in vivo. A method enabling simultaneous tracking of catheter tips and imaging of the deforming tissue/target is can improve placement accuracy and, thereby, eliminate the need to overcompensate with insertion of additional catheters to prevent underdosage of tumors resulting from a geographical miss.

To this end, a pulse sequence can be adapted to simultaneous track a reference location and target location(s) during an interventional procedure. Referring to FIG. 4, a non-limiting example of a pulse sequence that has these capabilities is shown. The example pulse sequence include four blocks occurring within one effective repetition time (“TR”):a tracking block 402, a steady state preparation block 404, an imaging sequence block 406, and a spoiling gradient block 408.

In the tracking block 402, magnetic field gradients are applied to sample spatial position (e.g., the x-, y-, and z-positions) of a tracking RF coil that is coupled to the medical device being used for the interventional procedure (e.g., a catheter). As a non-limiting example, the tracking RF coil can be a micro RF coil. In some embodiments, more than one tracking RF coils can be coupled to the medical device. A combination of zero-phase-reference and Hadamard encoding can be applied to correct for B₀ inhomogeneities. Phase field dithering can be integrated to eliminate B₁ inhomogeneities induced by the medical device.

Tracking data are acquired during the tracking block 402. From the tracking data, the position(s) of the tracking coil(s) can be determined. From this information, the position of the medical device (e.g., the reference location) can be known or determined. For instance, the position of tracking RF coils can be determined with peak positions detected using a centroid algorithm.

The imaging sequence block 406 can implement a slab selective, 3D balanced steady-state free precession (“bSSFP”) excitation. Data can be acquired using a Cartesian readout, or other suitable readout trajectory. Prior to the imaging sequence block 406, a steady-state preparation block 404 is applied. The steady-state preparation block 404 can include a non-linear ramp up of several RF pulses (e.g., eight consecutive RF pulses) that are played prior to the excitation RF pulse used in the imaging sequence block 406. As an alternative example, a fast spoiled gradient recalled echo (“FSPGR”) pulse sequence can be used in the imaging sequence block 406 to reduce banding artifacts that might otherwise interfere with image registration (e.g., deformable image registration) of target contours. In these instances, the steady-state preparation block 44 for FSPGR implements a succession of dummy repetitions (e.g., 40 dummy repetitions), increasing the effective TR of the combined tracking/imaging sequence and decreasing the update rate. In some embodiments, tracking data can be updated at an update rate of 20 Hz or faster, and imaging data can be updated at an update rate of 0.25 Hz or faster.

Spoiling gradients are then applied in the spoiling gradient block 408 to dephase any remaining transverse magnetization prior to the next effective TR. It will be appreciated by those skilled in the art that when using tracking RF coils, detuning of imaging and tracking coils can be performed prior to tracking and imaging, respectively.

In some embodiments, dynamic alteration of imaging planes based on device position updates obtained from active tracking can be implemented. In applications for high dose rate interstitial brachytherapy (“HDR-IB”), altering imaging planes may not improve accuracy in the presence of catheter flexion and tissue deformation. In these instances, continued 3D imaging of the target can be used to incorporate tissue deformation information during catheter navigation.

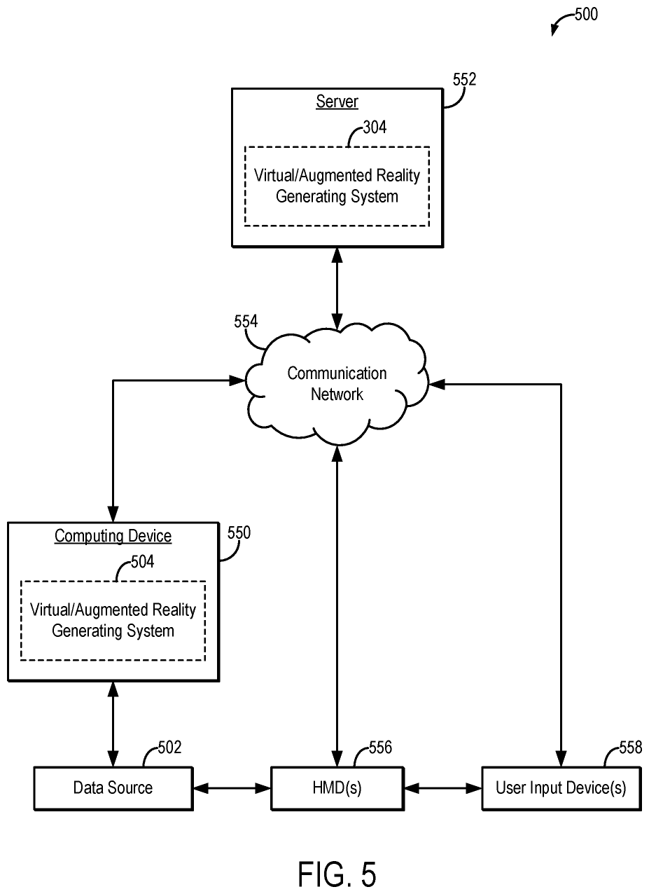

Referring now to FIG. 5, an example of a system 500 for generating and presenting a virtual/augmented reality environment to a user in accordance with some embodiments of the systems and methods described in the present disclosure is shown. As shown in FIG. 5, a computing device 550 can receive one or more types of data (e.g., image data, physiological data) from data source 502, which may be a magnetic resonance data source. In some embodiments, computing device 550 can execute at least a portion of a virtual/augmented reality environment generating system 504 to generate and present a virtual and/or augmented reality environment to a user based on data received from the data source 502.

Additionally or alternatively, in some embodiments, the computing device 550 can communicate information about data received from the data source 502 to a server 552 over a communication network 554, which can execute at least a portion of the virtual/augmented reality environment generating system 504. In such embodiments, the server 552 can return information to the computing device 550 (and/or any other suitable computing device) indicative of an output of the virtual/augmented reality environment generating system 504.

In some embodiments, computing device 550 and/or server 552 can be any suitable computing device or combination of devices, such as a desktop computer, a laptop computer, a smartphone, a tablet computer, a wearable computer, a server computer, a virtual machine being executed by a physical computing device, a head-mounted display, and so on. In some embodiments, a user can select content, upload content, etc., using the computing device 550 and/or the server 552 using any suitable technique or combination of techniques. For example, the computing device 550 can execute an application from memory that is configured to facilitate selection of medical imaging data to be presented, assembling the medical imaging data into a 3D array to be used in generating a 3D model, uploading the medical imaging data to a server (e.g., server 552) for distribution to one or more HMD(s) 556, downloading the medical imaging data to one or more HMD(s) 556, etc. The computing device 550 and/or server 552 can also reconstruct images from the data.

In some embodiments, the server 552 can be located locally or remotely from the HMD(s) 556. Additionally, in some embodiments, multiple servers 552 can be used (which may be located in different physical locations) to provide different content, provide redundant functions, etc.

In some embodiments, data source 502 can be any suitable source of image data (e.g., measurement data, images reconstructed from measurement data), such as an MRI system, any suitable source of physiological data (e.g., respiratory signal data, cardiac signal data, patient monitor data), another computing device (e.g., a server storing image data), and so on. In some embodiments, data source 502 can be local to computing device 550. For example, data source 502 can be incorporated with computing device 550 (e.g., computing device 550 can be configured as part of a device for capturing, scanning, and/or storing images). As another example, data source 502 can be connected to computing device 550 by a cable, a direct wireless link, and so on. Additionally or alternatively, in some embodiments, data source 502 can be located locally and/or remotely from computing device 550, and can communicate data to computing device 550 (and/or server 552) via a communication network (e.g., communication network 554).

In some embodiments, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 554 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, and so on.

In some embodiments, the computing device 550 and/or server 552 can provide and/or control content that is to be presented by one or more HMDs 556. In these instances, the computing device 550 and/or server 552 can communicate content to the HMD(s) 556 over the communication network 554. Additionally or alternatively, content can be communicated from the data source 502 to the HMD(s) 556 via a communications link. In some embodiments, the communications link can be any suitable communications link that can facilitate communication between the data source 502 and the HMD(s) 556. For example, communications link can be a wired link (e.g., a USB link, an Ethernet link, a proprietary wired communication link, etc.) and/or a wireless link (e.g., a Bluetooth link, a Wi-Fi link, etc.).

Additionally, in some embodiments, the system 500 can include one or more user input devices 558, which can communicate with the HMD(s) 556 via a communications link. In some embodiments, the communications link can be any suitable communications link that can facilitate communication between the user input device(s) 558 and the HMD(S) 556. For example, communications link 506 can be a wired link (e.g., a USB link, an Ethernet link, a proprietary wired communication link, etc.) and/or a wireless link (e.g., a Bluetooth link, a Wi-Fi link, etc.).

In some embodiments, the user input device(s) 558 can include any suitable sensors for determining a position of user input device 558 with respect to one or more other devices and/or objects (e.g., HMD(s) 558, a particular body part of a wearer of the HMD(s) 556, etc.), and/or a relative change in position (e.g., based on sensor outputs indicating that a user input device 558 has been accelerated in a particular direction, that a user input device 558 has been rotated in a certain direction, etc.). For example, in some embodiments, user input device 558 can include one or more accelerometers, one or more gyroscopes, one or more electronic compasses, one or more image sensors, an inertial measurement unit, or the like. In some embodiments, the user input device(s) 558 can be local to the HMD(s) 556.

In some embodiments, the user input device(s) 558 can be used as a pointing device by the wearer of the HMD(s) 556 to highlight a particular portion of content (e.g., to segment a portion of the images) being presented by the HMD(s) 556, to select a particular portion of the images (e.g., to control the orientation of the images, to control a position of the images, etc.), to control one or more user interfaces represented by the HMD(s) 556, and so on. Additionally, in some embodiments, representations of user input devices, surgical instruments, and so on, can be also be presented within the virtual/augmented reality environment.

In some embodiments, the HMD(s) 556, the server 552, and/or the computing device 550 can receive data from the user input device(s) 558 (e.g., via communication network 554) indicating movement and/or position data of the user input device(s) 558. Based on the data from the user input device(s) 558, the HMD(s) 556, the server 552, and/or the computing device 550 can determine one or more changes to the content being presented (e.g., a change in orientation and/or position of images, real objects, or virtual objects; a location of a contouring brush or other user interface tool for interacting with the virtual/augmented reality environment; one or more voxels that have been segmented or otherwise selected in the virtual/augmented reality environment; etc.).

In some embodiments, the user input device(s) 558 can be implemented using any suitable hardware. For example, the user input device(s) 558 can include one or more controllers that are configured to receive input via one or more hardware buttons, one or more touchpads, one or more touchscreens, one or more software buttons, etc. As another example, the user input device(s) 558 can include one or more controllers that are configured to receive input via translation and/or rotation along and around various axes, such as a six degrees-of-freedom (“DOF”) controller.

In some embodiments, the user input device(s) 558 can be an integral part of the HMD(s) 556, which can, for example, determine a direction in which a particular HMD 556 is pointing with respect to a virtual/augmented reality environment and/or real/virtual object(s). The information about which direction the HMD 556 is pointing can be used to infer a direction in which the wearer's eyes are looking (which can, for example, be augmented based on gaze information, in some cases). In some embodiments, the inferred location at which the wearer of HMD 556 is looking can be used as input to position one or more user interface elements with respect to the virtual environment and/or virtual object, and/or to control an orientation, magnification, and/or position at which to present a virtual object (e.g., as the direction in which a user looks changes, the HMD 556 can change how content is rendered to allow a user to move around an object as though the object were physically present in front of the user).

In some embodiments, the user input device(s) 558 can be a separate device, or devices, that can convey location information and/or movement information to the HMD(s) 556, the server 552, and/or the computing device 550, which can then be used to generate on or more user interface elements (e.g., representations of the user input device(s) 558), to facilitate user interaction with the virtual environment being presented via the HMD(s) 556, and/or virtual object(s) in the virtual environment.

In some embodiments, a user can interact with the computing device 550 and/or the server 552 to select content that is to be presented by the HMD(s) 556 (e.g., a particular scan to be presented). For example, the user can instruct the computing device 550 and/or the server 552 to send the HMD(s) 556 images corresponding to a particular volumetric medical imaging scan (e.g., MRI scan, CT scan, etc.). Additionally or alternatively, in some embodiments, the user can log-in to an application executed by the HMD(s) 556, and/or a service provided via the HMD(s) 556, using the computing device 550 and/or server 552.

In some embodiments, the user can generate a virtual scene to be presented by the HMD(s) 556 via the computing device 550, and/or the server 520. For example, a user can select imaging data to be used, one or more surgical instruments that are to be made available (e.g., for planning a surgical intervention), one or more patient models to be displayed, one or more radiation treatment plans or associated data (e.g., treatment contours, OAR contours), etc. As another example, in some embodiments, a user can use a conventional DICOM viewer to perform a segmentation of the imaging information (e.g., a user that does not have access to the mechanisms described herein), and can cause the segmentation to be associated with the volumetric medical imaging data. In such an example, such a segmentation can be used by the HMD(s) 556 to present the segmentation with a 3D model generated from the volumetric medical imaging data.

In some embodiments, the user can upload content and/or identifying information of content to the server 552 that is to be presented by the HMD(s) 556 from the computing device 550. For example, the user can upload volumetric medical imaging data, information about a surgical tool (e.g., dimensions, materials, color(s), etc.), information about a radiation source (e.g., dimensions, operating parameters, power, etc.), and/or any other suitable information that can be used in connection with some embodiments of the disclosed subject matter. As another example, the user can provide location information (e.g., a URL) at which content to be presented can be accessed. In some embodiments, the HMD(s) 556 can download and/or save the content at any suitable time. For example, the user or an administrator can download, sideload, and/or otherwise transfer the content to be viewed to each HMD 556.

In some embodiments, each HMD 556 can execute an application that can use medical imaging data to present a 3D model of a patient based on the medical imaging scan. In some embodiments, a user of the HMD(s) 556 can control presentation of the content in the virtual/augmented reality environment by providing input to the HMD(s) 556. For example, the HMD(s) 556 can be designated as having control of the virtual/augmented reality environment and/or one or more objects within the virtual/augmented reality environment.

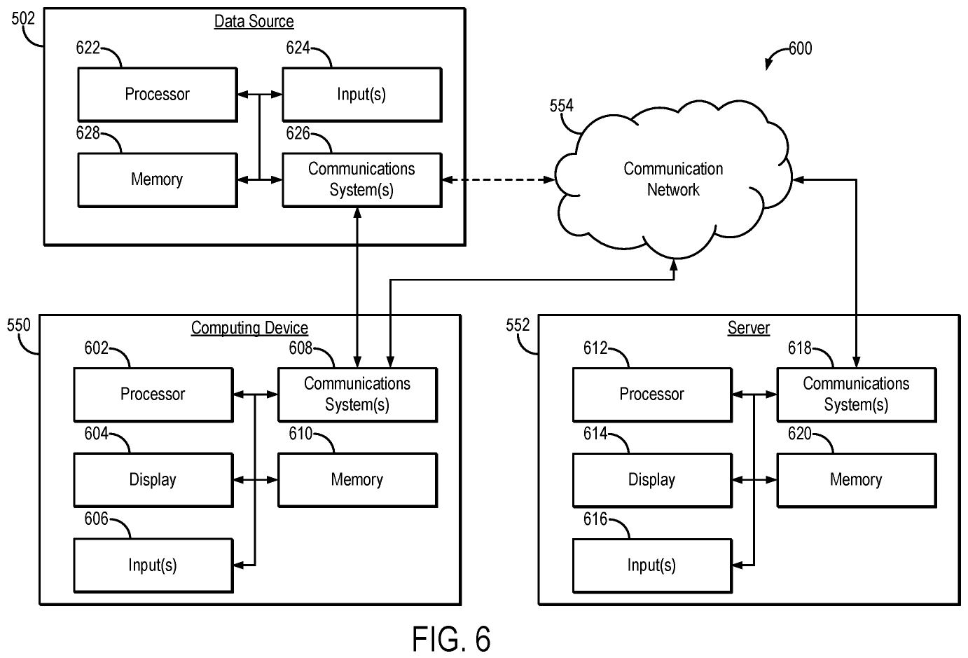

Referring now to FIG. 6, an example of hardware 600 that can be used to implement data source 502, computing device 550, and server 552 in accordance with some embodiments of the systems and methods described in the present disclosure is shown. As shown in FIG. 6, in some embodiments, computing device 550 can include a processor 602, a display 604, one or more inputs 606, one or more communication systems 608, and/or memory 610. In some embodiments, processor 602 can be any suitable hardware processor or combination of processors, such as a central processing unit (“CPU”), a graphics processing unit (“GPU”), and so on. In some embodiments, display 604 can include any suitable display devices, such as a computer monitor, a touchscreen, a television, and so on. In some embodiments, inputs 606 can include any suitable input devices and/or sensors that can be used to receive user input, such as a keyboard, a mouse, a touchscreen, a microphone, and so on.

In some embodiments, communications systems 608 can include any suitable hardware, firmware, and/or software for communicating information over communication network 554 and/or any other suitable communication networks. For example, communications systems 608 can include one or more transceivers, one or more communication chips and/or chip sets, and so on. In a more particular example, communications systems 608 can include hardware, firmware and/or software that can be used to establish a Wi-Fi connection, a Bluetooth connection, a cellular connection, an Ethernet connection, and so on.

In some embodiments, memory 610 can include any suitable storage device or devices that can be used to store instructions, values, data, or the like, that can be used, for example, by processor 602 to present content using display 604, to communicate with server 552 via communications system(s) 608, and so on. Memory 610 can include any suitable volatile memory, non-volatile memory, storage, or any suitable combination thereof. For example, memory 610 can include RAM, ROM, EEPROM, one or more flash drives, one or more hard disks, one or more solid state drives, one or more optical drives, and so on. In some embodiments, memory 610 can have encoded thereon, or otherwise stored therein, a computer program for controlling operation of computing device 550. In such embodiments, processor 602 can execute at least a portion of the computer program to present content (e.g., images, user interfaces, graphics, tables), receive content from server 552, transmit information to server 552, and so on.

In some embodiments, server 552 can include a processor 612, a display 614, one or more inputs 616, one or more communications systems 618, and/or memory 620. In some embodiments, processor 612 can be any suitable hardware processor or combination of processors, such as a CPU, a GPU, and so on. In some embodiments, display 614 can include any suitable display devices, such as a computer monitor, a touchscreen, a television, and so on. In some embodiments, inputs 616 can include any suitable input devices and/or sensors that can be used to receive user input, such as a keyboard, a mouse, a touchscreen, a microphone, and so on.

In some embodiments, communications systems 618 can include any suitable hardware, firmware, and/or software for communicating information over communication network 554 and/or any other suitable communication networks. For example, communications systems 618 can include one or more transceivers, one or more communication chips and/or chip sets, and so on. In a more particular example, communications systems 618 can include hardware, firmware and/or software that can be used to establish a Wi-Fi connection, a Bluetooth connection, a cellular connection, an Ethernet connection, and so on.

In some embodiments, memory 620 can include any suitable storage device or devices that can be used to store instructions, values, data, or the like, that can be used, for example, by processor 612 to present content using display 614, to communicate with one or more computing devices 550, and so on. Memory 620 can include any suitable volatile memory, non-volatile memory, storage, or any suitable combination thereof. For example, memory 620 can include RAM, ROM, EEPROM, one or more flash drives, one or more hard disks, one or more solid state drives, one or more optical drives, and so on. In some embodiments, memory 620 can have encoded thereon a server program for controlling operation of server 552. In such embodiments, processor 612 can execute at least a portion of the server program to transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone), and so on.

In some embodiments, data source 502 can include a processor 622, one or more input(s) 624, one or more communications systems 626, and/or memory 628. In some embodiments, processor 622 can be any suitable hardware processor or combination of processors, such as a CPU, a GPU, and so on. In some embodiments, the one or more input(s) 624 are generally configured to acquire data, images, or both, and can include an MRI system, other medical imaging system, and/or a physiological monitoring system (e.g., respiratory bellows, electrocardiography system, other patient monitor). Additionally or alternatively, in some embodiments, one or more input(s) 624 can include any suitable hardware, firmware, and/or software for coupling to and/or controlling operations of an MRI system, other medical imaging system, and/or a physiological monitoring system. In some embodiments, one or more portions of the one or more input(s) 624 can be removable and/or replaceable.

Note that, although not shown, data source 502 can include any suitable inputs and/or outputs. For example, data source 502 can include input devices and/or sensors that can be used to receive user input, such as a keyboard, a mouse, a touchscreen, a microphone, a trackpad, a trackball, and so on. As another example, data source 502 can include any suitable display devices, such as a computer monitor, a touchscreen, a television, etc., one or more speakers, and so on.

In some embodiments, communications systems 626 can include any suitable hardware, firmware, and/or software for communicating information to computing device 550 (and, in some embodiments, over communication network 554 and/or any other suitable communication networks). For example, communications systems 626 can include one or more transceivers, one or more communication chips and/or chip sets, and so on. In a more particular example, communications systems 626 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 628 can include any suitable storage device or devices that can be used to store instructions, values, data, or the like, that can be used, for example, by processor 622 to control the one or more input(s) 624, and/or receive data from the one or more input(s) 624; to images from data; present content (e.g., images, a user interface) using a display; communicate with one or more computing devices 550; and so on. Memory 628 can include any suitable volatile memory, non-volatile memory, storage, or any suitable combination thereof. For example, memory 628 can include RAM, ROM, EEPROM, one or more flash drives, one or more hard disks, one or more solid state drives, one or more optical drives, and so on. In some embodiments, memory 628 can have encoded thereon, or otherwise stored therein, a program for controlling operation of data source 502. In such embodiments, processor 622 can execute at least a portion of the program to generate images, transmit information and/or content (e.g., data, images) to one or more computing devices 550, receive information and/or content from one or more computing devices 550, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

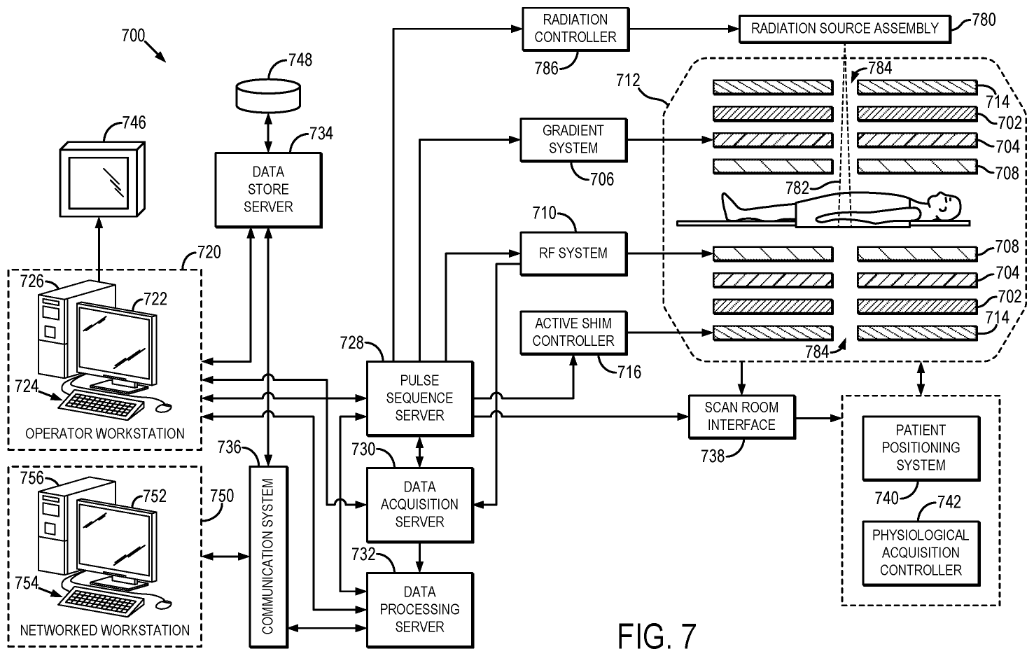

Referring particularly now to FIG. 7, an example of an MRI system 700 that can implement the methods described here is illustrated. In the configuration shown in FIG. 7, the MRI system 700 incorporates a linear accelerator (“linac”) for providing radiation treatment to a patient. In this way, the MRI system 700 may be referred to as an MR-Linac system.

The MRI system 700 includes a magnet assembly 702 that generates a main magnetic field, B₀, which may also be referred to as a polarizing magnetic field. The MRI system 700 also includes a gradient coil assembly 704 containing one or more gradient coils, which is controlled by a gradient system 706, and a radiofrequency (“RF”) coil assembly 708 containing one or more RF coils, which is controlled by an RF system 710.

The RF coil assembly 708 can include one or more RF coils that are enclosed within a housing 712 of the MRI system 700, or can include one or more RF coils that are physically separate from the housing 712, such as local RF coils that can be interchangeably positioned within the bore of the MRI system 700. Similarly, the gradient coil assembly 704 can include one more gradient coils that are enclosed within the housing 712 of the MRI system 700, or can include one or more gradient coils that are physically separate from the housing 712 and that can be interchangeably positioned within the bore of the MRI system 700. The housing 712 may be sized to receive a subject's body, or sized to receive only a portion thereof, such as a subject's head.

The magnet assembly 702 generally includes a superconducting magnet that is formed as one or more magnet coils made with superconducting wire, high temperature superconducting (“HTS”) wire, or the like. The one or more magnet coils can be arranged as a solenoid, a single-sided magnet, a dipole array, or other suitable configuration. The superconducting magnet can be cooled using a liquid or gaseous cryogen. In some other configurations, the magnet assembly 702 can include one or more electromagnets, resistive magnets, or permanent magnets. For example, the magnet assembly 702 could include a Halbach array of permanent magnets.

As will be described, the RF coil assembly 708 generates one or more RF pulses that rotate magnetization of one or more resonant species in a subject or object positioned in the main magnetic field, B₀, generated by the magnet assembly 702. In response to the one or more transmitted RF pulses, magnetic resonance signals are generated, which are detected to form an image of the subject or object. The gradient coil assembly 704 generates magnetic field gradients for spatially encoding the magnetic resonance signals. Collectively, the one or more RF pulses and the one or more magnetic field gradients define a magnetic resonance pulse sequence.

In some configurations, the MRI system 700 can also include a shim coil assembly 714. The shim coil assembly 714 can include passive shims, active shims, or combinations thereof. Active shims can include active shim coils that generate magnetic fields in order to shim, or reduce inhomogeneities, in the main magnetic field, B₀, generated by the magnet assembly 702. In some configurations, the active shim coils are controlled by an active shim controller 716.

The MRI system 700 includes an operator workstation 720 that may include a display 722, one or more input devices 724 (e.g., a keyboard, a mouse), and a processor 726. The processor 726 may include a commercially available programmable machine running a commercially available operating system. The operator workstation 720 provides an operator interface that facilitates entering scan parameters into the MRI system 700. The operator workstation 720 may be coupled to different servers, including, for example, a pulse sequence server 728, a data acquisition server 730, a data processing server 732, and a data store server 734. The operator workstation 720, the pulse sequence server 728, the data acquisition server 730, the data processing server 732, and the data store server 734 may be connected via a communication system 736, which may include wired or wireless network connections.

The pulse sequence server 728 functions in response to instructions provided by the operator workstation 720 to operate the gradient system 706 and the RF system 710. Gradient waveforms for performing a prescribed scan are produced and applied to the gradient system 706, which then excites gradient coils in the gradient coil assembly 704 to produce the magnetic field gradients (e.g., Gₓ, G_{y}, and G_{z} gradients) that are used for spatially encoding magnetic resonance signals.

RF waveforms are applied by the RF system 710 to the RF coil assembly 708 to generate one or more RF pulses in accordance with a prescribed magnetic resonance pulse sequence. Magnetic resonance signals that are generated in response to the one or more transmitted RF pulses are detected by the RF coil assembly 708 and received by the RF system 710. The detected magnetic resonance signals may be amplified, demodulated, filtered, and digitized under direction of commands produced by the pulse sequence server 728.

The RF system 710 includes an RF transmitter for producing a wide variety of RF pulses used in magnetic resonance pulse sequences. The RF transmitter may include a single transmit channel, or may include multiple transmit channels each controlling a different RF transmit coil. The RF transmitter is responsive to the prescribed scan and direction from the pulse sequence server 728 to produce RF pulses of the desired frequency, phase, and pulse amplitude waveform. The generated RF pulses may be applied to the RF coil assembly 708, which as described above may include one or more RF coils enclosed in the housing 712 of the MRI system 700 (e.g., a body coil), or one or more RF coils that are physically separate from the housing 712 (e.g., local coils or coil arrays).

The RF system 710 also includes one or more RF receiver channels. An RF receiver channel includes an RF preamplifier that amplifies the magnetic resonance signal received by the RF coil in the RF coil assembly 708 to which the receiver channel is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} ; \qquad ( 1 )$$

and the phase of the received magnetic resonance signal may also be determined according to the following relationship:

$$φ = {tan}^{- 1} ( \frac{Q}{I} ) . \qquad ( 2 )$$

When the MRI system 700 includes a shim assembly 714 having one or more active shim coils, the pulse sequence server 728 can also connect to an active shim controller 716 to apply shim coil waveforms for generating magnetic fields to shim the main magnetic field, B₀, generated by the magnet assembly 702.

The pulse sequence server 728 may also connect to a scan room interface 738 that can receive signals from various sensors associated with the condition of the subject or object being imaged, the magnet assembly 702, the gradient coil assembly 704, the RF coil assembly 708, the shim assembly 714, or combinations thereof. In one example, the scan room interface 738 can include one or more electrical circuits for interfacing the pulse sequence server 728 with such sensors. Through the scan room interface 738, a patient positioning system 740 can receive commands to move the subject or object being imaged to desired positions during the scan, such as by controlling the position of a patient table.

The pulse sequence server 728 may also receive physiological data from a physiological acquisition controller 742 via the scan room interface 738. By way of example, the physiological acquisition controller 742 may receive signals from a number of different sensors connected to the subject, including electrocardiograph (“ECG”) signals from electrodes, respiratory signals from a respiratory bellows or other respiratory monitoring devices, and so on. These signals may be used by the pulse sequence server 728 to synchronize, or “gate,” the performance of the scan with the subject's heart beat or respiration.

Digitized magnetic resonance signal samples produced by the RF system 710 are received by the data acquisition server 730 as magnetic resonance data, which may include k-space data. In some scans, the data acquisition server 730 passes the acquired magnetic resonance data to the data processing server 732. In scans that implement information derived from the acquired magnetic resonance data to control further performance of the scan, the data acquisition server 730 may be programmed to produce such information and to convey it to the pulse sequence server 728. For example, during pre-scans, magnetic resonance data may be acquired and used to calibrate the pulse sequence performed by the pulse sequence server 728. As another example, navigator signals may be acquired and used to adjust the operating parameters of the RF system 710 or the gradient system 706, or to control the view order in which k-space is sampled.

The data processing server 732 receives magnetic resonance data from the data acquisition server 730 and processes the magnetic resonance data in accordance with instructions provided by the operator workstation 720. Such processing may include, for example, reconstructing two-dimensional or three-dimensional images by performing a Fourier transformation of raw k-space data, performing other image reconstruction algorithms (e.g., iterative reconstruction algorithms), applying filters to raw k-space data or to reconstructed images, and so on.

Images reconstructed by the data processing server 732 can be conveyed back to the operator workstation 720 for storage. Real-time images may be stored in a data base memory cache, from which they may be output to operator display 722 or to a separate display 746. Batch mode images or selected real-time images may also be stored in a data storage 748, which may be a host database containing a disc storage. When such images have been reconstructed and transferred to storage, the data processing server 732 may notify the data store server 734 on the operator workstation 720. The operator workstation 720 may be used by an operator to archive the images, produce films, or send the images via a network to other facilities.

The MRI system 700 may also include one or more networked workstations 750. For example, a networked workstation 750 may include a display 752, one or more input devices 754 (e.g., a keyboard, a mouse), and a processor 756. The networked workstation 750 may be located within the same facility as the operator workstation 720, or in a different facility, such as a different healthcare institution or clinic.

The networked workstation 750 may gain remote access to the data processing server 732 or data store server 734 via the communication system 736. Accordingly, multiple networked workstations 750 may have access to the data processing server 732 and the data store server 734. In this manner, magnetic resonance data, reconstructed images, or other data may be exchanged between the data processing server 732 or the data store server 734 and the networked workstations 750, such that the data or images may be remotely processed by a networked workstation 750.

The MRI system 700 also includes a radiation source assembly 780 that is coupled to the housing 712 of the MRI system 700. As one example, the radiation source assembly 780 can include a gantry onto which a radiation source (e.g., a linear accelerator) is mounted. The radiation source assembly 780 generates a radiation beam that is directed towards a patient positioned in the bore of the MRI system 700 to provide radiation treatment to that patient.

In the configuration shown in FIG. 7, the magnet assembly 702, gradient coil assembly 704, RF coil assembly 708, and shim coil assembly 714 can each be split assemblies in order to define a space 784 through which the radiation beam 782 generated by the radiation source assembly 760 can be delivered to reach the patient.

The radiation source assembly 780 is controlled by a radiation controller 786. As an example, the radiation controller 786 can control the rotation of the gantry, such that the position of the radiation source is moved about the perimeter of the housing 712 of the MRI system 700 into different angular orientation. The radiation controller 786 also controls turning the radiation beam 782 on and off according to a prescribed radiation treatment plan, or in response to other control signals, instructions, or plans. In some instances, the radiation controller 786 may receive instructions and/or data from the pulse sequence server 728, such that the radiation beam 782 may be turned on and off in conjunction with, or relative to, a prescribed pulse sequence. Additionally or alternatively, the radiation controller 786 may receive data from the physiological acquisition controller 742, which may also be used to control turning the radiation beam on and off, such as relative to a patient's cardiac motion, respiratory motion, or both.

The present disclosure has described one or more preferred embodiments, and it should be appreciated that many equivalents, alternatives, variations, and modifications, aside from those expressly stated, are possible and within the scope of the invention.

1. A method for image-guided alignment of an interventional device, comprising:
(a) accessing medical image data with a computer system, wherein the medical image data are acquired from a subject in real-time with a medical imaging system, wherein the medical image data depict an anatomical target;
(b) generating with the computer system, an augmented reality environment based in part on the medical image data, wherein the augmented reality environment comprises at least one visual guide indicating a separation distance between a reference location and the anatomical target, wherein generating the augmented reality environment comprises overlaying the at least one visual guide with a view of the subject in a real-world environment;
(c) displaying the augmented reality environment to a user using a display while medical image data continue to be acquired in real-time from the subject with the medical imaging system; and
(d) generating via the at least one visual guide in the augmented reality environment, an indication when the reference location is aligned with the anatomical target.

2. The method of claim 1, wherein the at least one visual guide comprises at least a first visual guide indicating a first separation distance between the reference location and the anatomical target along a first spatial dimension and a second visual guide indicating a second separation distance between the reference location and the anatomical target along a second spatial dimension that is orthogonal to the first spatial dimension.

3. The method of claim 2, wherein the at least one visual guide further comprises a third visual guide indicating a third separation distance between the reference location and the anatomical target along a third spatial dimension that is orthogonal to the first and second spatial dimensions.

4. The method of claim 1, wherein the at least one visual guide comprises a linear display element having a length that varies proportionally with the separation distance.

5. The method of claim 1, wherein the at least one visual guide comprises a linear display element having a color that varies based on the magnitude of the separation distance.

6. The method of claim 5, wherein the color of the linear display element varies between a first color when the separation distance is below a lower threshold, a second color when the separation distance is at or above the lower threshold and below an upper threshold, and a third color when the separation distance is above the upper threshold.

7. The method of claim 6, wherein the lower threshold is 0.5 cm and the upper threshold is 1 cm.

8. The method of claim 1, wherein the medical imaging system is a magnetic resonance imaging (MRI) system, wherein the reference location corresponds to a location on a medical device, and further comprising accessing tracking data acquired from a tracking radio frequency (RF) coil coupled to the medical device.

9. The method of claim 8, wherein the medical image data and the tracking data are acquired with the MRI system using a pulse sequence that in each repetition time (TR) period comprises:
a tracking sequence block in which the tracking data are acquired;
a steady-state preparation block in which a steady-state magnetization is generated; and
an imaging sequence block in which the medical image data are acquired.

10. The method of claim 9, wherein the imaging sequence block comprises a balanced steady-state free precession (bSSFP) acquisition.

11. The method of claim 9, wherein the imaging sequence block comprises a fast spoiled gradient recalled echo (FSPGR) acquisition.

12. The method of claim 1, wherein the reference location corresponds to a treatment isocenter of a radiation therapy system.

13. The method of claim 1, wherein the reference location corresponds to a tip of a medical device.

14. A method for controlling the delivery of radiation treatment, the method comprising:
(a) accessing medical images of a subject with a computer system, wherein the medical images are acquired in real-time with a medical imaging system, wherein the medical images depict an anatomical target;
(b) accessing treatment contour data with the computer system;
(c) generating with the computer system, a virtual reality environment using the medical images and the treatment contour data, wherein the virtual reality environment depicts a scene in which the treatment contour data are overlaid with the medical images;
(d) displaying the virtual reality environment to the subject using a display while medical images continue to be acquired in real-time from the subject with the medical imaging system; and
(e) triggering a radiation treatment system to turn on a radiation beam when the anatomical target is aligned within a contour of the treatment contour data within the virtual environment.

15. The method of claim 14, wherein the virtual reality environment comprises an augmented reality environment.

16. The method of claim 14, wherein the display is a head-mounted display worn by the subject.

17. The method of claim 14, wherein the medical imaging system is a magnetic resonance imaging (MRI) system.

18. The method of claim 17, wherein the radiation treatment system is integrated with the MRI system.

19. A method for image-guided alignment of an interventional device, comprising:
(a) accessing medical images of a subject with a computer system, wherein the medical images are acquired in real-time with a medical imaging system, wherein the medical images depict an anatomical target;
(b) generating with the computer system, an augmented reality environment using the medical images, wherein the augmented reality environment depicts a scene in which the medical images are overlaid with a view of the subject in a real-world environment;
(c) displaying the augmented reality environment to a user using a display while medical images continue to be acquired in real-time from the subject with the medical imaging system; and
(d) based on the augmented reality environment, aligning an interventional device with the anatomical target.

20. The method of claim 19, wherein the interventional device is a radiation treatment system and aligning the interventional device with the anatomical target comprises aligning a radiation beam of the radiation treatment system with the anatomical target.

21. The method of claim 19, wherein the interventional device is a needle and aligning the interventional with the anatomical target comprises aligning the needle with the anatomical target.

22. The method of claim 21, wherein the needle is a needle for delivering brachytherapy seeds.

23. The method of claim 21, wherein the needle is a biopsy needle.

24. A method for aligning a subject with a radiation beam of a radiation treatment system, comprising:
(a) accessing patient model data with a computer system;
(b) generating with the computer system, an augmented reality environment using the patient model data, wherein the augmented reality environment depicts a scene in which the patient model data are overlaid with a real-world environment;
(c) displaying the augmented reality environment to a user using a display; and
(d) generating an indication in the augmented reality environment when the patient model is aligned with a radiation beam of a radiation treatment system within the scene.
